(19) United States
(12) Patent Application Publication
(10) Pub. No.: US 2021/0155924 A1
(43) Pub. Date: May 27, 2021

(54) METHODS AND COMPOSITIONS FOR INDUCING TUMOR CELL DEATH

(71) Applicant: Stitch Bio, LLC, Waltham, MA (US)

(72) Inventor: Anthony P. Shuber, Northbridge, MA (US)

(21) Appl. No.: 17/104,607

(22) Filed: Nov. 25, 2020

Related U.S. Application Data
(60) Provisional application No. 62/941,027, filed on Nov. 27, 2019.

(51) Int. Cl. C12N 15/11; C12Q 1/6886; C12N 9/22; A61K 9/51; A61K 38/46; A61K 31/7088

(52) U.S. Cl. CPC C12N 15/11; C12Q 1/6886; C12N 9/22; A61K 9/5123; C12Q 2600/156; A61K 31/7088; C12N 2310/20; C12N 2800/80; C12N 2320/32; A61K 38/465

(57) ABSTRACT

The disclosure provides methods and compositions that employ gene editing systems to enable cells to express guide RNAs. Gene editing systems specifically target fusions in tumor DNA to introduce a coding sequence that is expressed by tumor cells as a guide RNA that targets known repetitive elements in the human genome in tumor cells. The CRISPR-like systems are expressed in the tumor cells and cleave the tumor DNA at the known repetitive elements thereby inducing tumor cell death.

### TECHNICAL FIELD

The disclosure relates to methods and compositions for inducing tumor cell death.

### BACKGROUND

Personalized therapies focused on determining unique molecular characteristics of individual patients have become the forefront of research efforts. CRISPR (clustered regularly interspaced palindromic repeats)-Cas systems found in bacteria have revolutionized the field of genomic-based therapy allowing for simple, timesaving, and cost-efficient genome editing. CRISPR is a defense mechanism present in bacteria that provides a defense against (primarily) viruses. Viruses infect bacteria cells by binding to surface proteins and inserting their DNA through the cell wall, where the cell then replicates the viral DNA. Bacteria cells store small fragments of the viral DNA, known as guide RNAs, in their genome for future comparison to foreign DNA. If the sequences of the guide RNA (gRNA) and foreign DNA match, a CRISPR-associated (Cas) protein cleaves the foreign DNA. By virtue of the sequence of the gRNA, a CRISPR-Cas complex cleaves target genetic material in a specific and controllable manner. Thus, CRISPR-Cas systems can be used to edit single or multiple genes to treat inherited disorders, cancer, and viral infections.

Gene editing therapies have not found a workable application in cancer and continue to suffer from off-target binding gene. For example, off-target binging can lead to genomic instability and may disrupt otherwise normal genes.

### SUMMARY

The invention utilizes RNA-guided Cas endonucleases to selectively target cancer cells. In particular, the invention provides compositions and methods that include vectors that target DNA fusions present only in cancer cells for the expression of Cas or Cas-related endonucleases and guide RNA that target known repetitive elements in the human genome. Expression of the endonuclease and its associated guide RNA results in destruction of the genome of the cancer cell. Thus, the invention achieves specificity of action against cancer cells (via cancer-specific fusions) while targeting multiple known repetitive sequences for massive destruction of DNA—which leads to cell death.

Fusions are a hallmark of cancer. The specific fusions in any given cancer are unique to that cancer. The invention contemplates identifying one or more fusions in a tumor and designing vectors that target the fusions for incorporation of DNA. The DNA, when expressed, results in Cas-related endonucleases and RNAs that are associated with the endonucleases and act as guide RNAs (gRNAs) to target other elements in the genome of the tumor cells that are unlikely to be altered, such as known repeat elements. Even if some of the targeted repeat elements are altered in the cancer cells, the cells will still be destroyed, as the invention contemplates targeting multiple repeat sites. Once the nuclease acts on targeted regions, the DNA will be destroyed and the cells will undergo apoptosis or necrosis.

Accordingly, the invention provides methods, systems, and compositions for treating cancer. The invention relies on expression of gRNAs and Cas-related endonucleases in tumor cells by targeting fusions found only in tumor genomes. Methods of the invention include delivering vectors comprising DNA encoding Cas-related endonucleases and DNA encoding gRNAs complimentary to known repeats in the human genome to tumor cells. Upon targeted delivery into tumor cells, the Cas-related endonucleases and gRNAs are expressed. The exogenous gRNAs bind at or near known repeat sequences in tumor cells and Cas-related endonucleases cleave the known repeat sequences therein, resulting in fragmentation of tumor DNA. In one alternative, the Cas protein can be delivered as a protein rather than being encoded in DNA. Fragmentation of tumor DNA results in the destruction of the tumor cells, whether by apoptosis, or simply by destroying the cells from the inside out. Thus, methods and systems of the invention treat cancer by inducing tumor cell death by expressing CRISP-like systems within the tumor cells.

Methods of the invention include inducing tumor cell death using systems of the invention. The systems of the invention may include a genome-editing tool such as a Cas endonuclease, or nucleic acid encoding the Cas endonuclease, including gRNAs that target a fusion in the tumor genome. The systems may also include coding sequences for guide RNAs complimentary to known repetitive sequences, which may be provided in an expression vector (e.g., an expression cassette). The genome editing tools selectively target a tumor genome by virtue of being designed to act on sequences found specifically in the tumor genome and not also in corresponding portions of matched normal sequences from the same patient or subject. In the tumor cells, the genome editing tools target and cleave the tumor-specific sequences, resulting in insertion and integration of the exogenous coding sequences, e.g., by homology-directed end repair, into the tumor genome. The exogenous coding sequences may be provided as an expression cassette with regulatory sequences (e.g., expression control sequences) such as promoters or transcription factor binding sites that induce expression of those coding sequences as gRNAs for association with the expressed Cas-related endonucleases in the tumor cells that function as a CRISPR-like system within the tumor cells only.

The systems of the invention may include recombinant DNA molecules adapted for expression of a gRNA for association with Cas-related endonucleases within the tumor cell. The recombinant DNA molecules may include nucleic acid molecules encoding a gRNA complementary to repetitive elements in the human genome. The system may also include recombinant DNA molecules adapted for expression of a Cas-related endonuclease for association with the gRNA. The Cas-endonuclease recombinant molecules may include nucleic acid molecules encoding Cas-related endonucleases. The gRNA recombinant DNA molecule may also have expression control sequences operatively linked thereto. The expression control sequences may be sequences of promoters or transcription factor binding sites that induce expression of those nucleic acid molecules encoding the gRNAs. Once expressed in the tumor cells, the gRNAs and the Cas-related endonucleases cleave known repeat sequences in the tumor DNA.

The recombinant DNA molecules may include bacterial or viral DNA. The recombinant DNA molecules may be viral vectors containing the nucleic acid molecules encoding Cas-related endonuclease, the nucleic acid molecules encoding gRNA, or both. The recombinant DNA molecules may be in combination with each other and provided together in vectors. The vectors containing the Cas-related endonuclease recombinant DNA molecules may also have fusion-specific moieties operatively linked thereto. The fusion-specific moieties may target the vectors containing the recombinant DNA molecules to cells expressing the fusions, thereby directing expression of at least the gRNA only in tumor cells. Alternatively, gene editing systems that target one or more fusions may be used to insert the gRNA sequences at the one or more fusions to cause expression of the gRNA within tumor cells only.

Methods may include identifying sequences found specifically in a tumor genome and not in corresponding portions of matched normal sequences from the same patient and designing vectors to target those tumor-specific genomic sequences. Identifying tumor-specific sequences, such as fusions, may include obtaining a patient sample and analyzing tumor DNA sequences from the sample to identify sequences that are in the tumor DNA but not also present in matched-normal DNA from the patient. For example, patient samples may be obtained that include tumor and non-tumor cells from any suitable source including germline or somatic sources. Sequencing may be performed, e.g., using next-generation sequencing instruments, and resulting tumor sequences may be compared and matched to corresponding sequences from non-tumor cells, the “matched normal” sequences. Sequences appearing exclusively in the tumor genome may thus be identified as the targets suitable for targeting with Cas gene editing systems containing DNA encoding gRNAs complimentary to known repeats in the human genome. Delivering the exogenous coding sequences into tumor cells with Cas gene editing systems that exclusively target tumor cells, allows tumor cells to express CRISPR-like systems of the present invention that target and cleave known repeats in the human genome, thus destroying tumor cells.

Methods of the invention include using a Cas gene editing system to induce expression of gRNAs complimentary to one or more repetitive elements in the human genome in a tumor cell. The gene editing system delivered to the subject may include at least one Cas endonuclease or a nucleic acid encoding the Cas endonuclease. In some embodiments, the Cas endonucleases include one or more guide RNAs that target delivery of the coding sequence for the exogenous RNA to a predetermined site in the tumor genome. The predetermined site may include, for example, a genomic safe harbor. The gene editing system may include at least a ribonucleoprotein (RNP) that includes a Cas endonuclease and a guide RNA (gRNA) that binds the RNP to a predetermined site within the tumor-specific genomic material and introduces at least the coding sequence into the tumor-specific genomic material. The coding sequence may be provided in an expression cassette. The expression cassette may also introduce a promoter or a transcription factor binding site to increase transcription of the coding sequence, e.g., the gRNA complimentary to at least one repetitive element in the human genome. The nucleic acid sequence of the promoter or the transcription biding site may be included along with the nucleic acid sequence of a gRNA complimentary to at least one repetitive element in the human genome as a vector (e.g., expression cassette).

Methods of the invention include inducing tumor cell death using systems of the invention. The method may include identifying one or more fusion(s) in tumor DNA of a subject and delivering a first vector and a second vector to the subject. The first vector may include DNA encoding a guide RNA (gRNA) capable of hybridizing with a common region within a repetitive sequence present in the human genome. The second vector may include DNA encoding a Cas-related endonuclease. The first and second vectors may target one or more fusions identified in tumor DNA of a subject. The first and second vectors may be delivered to the subject simultaneously, or they may be delivered consecutively. The first and/or the second vector may include a gene editing system that targets the one or more fusions identified in the tumor DNA of the subject. The gene editing system may include a Cas-associated endonuclease and a gRNA (or set of guide RNAs) that target and cleave the one or more tumor-specific sequences (fusions), resulting in insertion and integration of the exogenous coding sequences, e.g., by homology-directed repair, into the tumor genome. The exogenous coding sequences may be provided as an expression cassette with regulatory sequences such as promoters or transcription factor binding sites that induce expression of those coding sequences as gRNAs complimentary to repetitive sequences in the human genome and/or Cas-associated endonucleases. Once expressed, the gRNAs associate with the Cas-associated endonucleases and function as CRISPR-like systems within the tumor cells. The gRNAs target the Cas-associated endonucleases to the repetitive sequences in tumor DNA and cleave the repetitive sequences therein.

The method may include obtaining tumor DNA from the subject and analyzing the tumor DNA (e.g., by sequencing or probe hybridization assays) to identify a fusion in the tumor DNA that is not found in matched normal sequences from healthy, non-tumor cells of the subject. Embodiments may include sequencing matched, normal DNA from the healthy, non-tumor cells of the subject to thereby obtain tumor sequences and matched normal sequences; aligning the tumor sequences to the matched normal sequences; and identifying the fusion as a section of the tumor sequence that does not have an exact match in the matched normal sequences.

The method may further include obtaining or synthesizing one or more guide RNAs with targeting portions that are complementary to the target in the tumor DNA when the target in the tumor DNA is adjacent a protospacer adjacent motif in the tumor DNA.

The method may further include obtaining or synthesizing one or more nucleic acid molecules encoding a gRNA with targeting portions that are complementary to a repetitive element in the human genome. The repetitive element may or may not be adjacent a protospacer adjacent motif in the tumor DNA. The nucleic acid molecules encoding gRNA with targeting portions complimentary to a repetitive element in the human genome may be included in an expression cassette. The expression cassette may be delivered to a tumor-specific site (e.g., a fusion) using a gene editing system of the present invention that targets the fusion sequence. That is, the expression vector (E.g., expression cassette) containing the nucleic acid molecules encoding a gRNA with targeting portions that are complementary to a repetitive element in the human genome, may be delivered, inserted, and thereby expressed in a tumor cell using a gene editing system with gRNAs that target the tumor-specific fusion.

In other aspects, the disclosure provides a composition that includes a gene editing system—or nucleic acid encoding the gene editing system—and an expression cassette. The gene editing system includes a targeting sequence that binds specifically to a target in a tumor genome and the expression cassette includes a coding sequence encoding a gRNA complementary to repetitive elements in the human genome. Preferably, the target in the tumor genome is not found in a genome from healthy, non-tumor cells of a subject with the tumor. When the composition is delivered to a subject, the gene editing system causes integration of the expression cassette into the tumor genome at the target. The integration results in expression of the coding sequence as a gRNA for association with a Cas-related endonuclease in a cell that includes the tumor genome. The expression results in the cleaving of repetitive elements in the tumor genome that includes the fusion.

In certain embodiments, the gene editing system includes a Cas-associated endonuclease and a gRNA that includes the targeting sequence, the Cas-associated endonuclease and gRNA may be complexed as a ribonucleoprotein (RNP). The Cas endonuclease, gRNA, an expression vector containing the nucleic acid encoding a gRNA complimentary to a known sequence in the human genome (e.g., a sequence of a known repetitive element), or all three may be packaged in one or more lipid particles for delivery, such as solid lipid nanoparticles or liposomes. Alternatively, the RNP, expression cassette, or both may be packed in one or more lipi particles for deliver. For example, the composition may include at least dozens, or several hundred, or several thousand of the solid lipid nanoparticles packaging at least a corresponding number of Cas-associated endonucleases, gRNAs, (or RNPs) and expression cassettes. The solid lipid nanoparticles may be packaged in a vessel or container such as a blood collection tube or a microcentrifuge tube. For example, in some embodiments, the container comprises a microcentrifuge tube. The solid lipid nanoparticles may be provided as an aqueous suspension in one or more such containers (e.g., with all tubes on optionally on dry ice in a Styrofoam container).

In related embodiments, the disclosure provides a kit that includes any of the foregoing compositions in one or more suitable containers.

The various methods, compositions, and kits of the disclosure are useful for inducing expression of CRISPR-like systems in a human cell. Compositions preferably include a Cas-associated gene editing system—or nucleic acid encoding the gene editing system—and nucleic acid encoding at least a segment of a gRNA corresponding to a sequence of a known repetitive element in a human genome. The composition may include the gene editing system as a Cas-associated endonuclease complexed with a guide RNA that specifically hybridizes to targets in a tumor genome. The Cas endonuclease and guide RNA may be present as a ribonucleoprotein (RNP). The nucleic acid encoding at least a segment of a gRNA corresponding to a sequence of a known repetitive element in a human genome may be an expression cassette for an exogenous coding sequence with one or more of a promoter and a transcription factor binding site, and—optionally—end segments that promote integration of the expression cassette into a tumor genome (e.g., by homology directed repair).

When the composition is introduced into a subject, the Cas-associated gene editing system targets the one or more fusions, delivering the nucleic acid encoding at least the gRNA into the tumor cell. Upon delivery of the nucleic acid, the tumor cell expresses the gRNA within the tumor cell only. Once expressed, the CRISPR-like system is activated, causing the gRNA to hybridize to corresponding segments of the known sequence repeats and the Cas-related endonuclease to cleave the tumor DNA. The Cas-associated gene editing system may specifically target sequences exclusive to a tumor genome that have been identified via methods of the disclosure. For example, the tumor-specific genomic material, i.e., the one or more fusions may be detected by comparing tumor sequences to “matched normal” sequences, either of which may be obtained by next generation sequencing technologies. The methods may also include sequencing DNA obtained from the subject's sample. Once a tumor cell is identified by Cas-associated gene editing system, the nucleic acid encoding the gRNA is inserted at the fusion site. Thus, the gRNA, though specific for known sequence repeats that occur in both tumor and normal cells, only hybridizes to those repeat sequences in the tumor cell because it is only expressed in the tumor cell. As such, the Cas-related endonuclease only cleaves tumor DNA and not normal DNA when expressed in the presence of the gRNA expressed in the tumor cell.

In certain embodiments, the Cas-associated gene editing system includes a first ribonucleoprotein (RNP) that includes a Cas endonuclease and a guide RNA (gRNA). The composition may include a second RNP. By virtue of the gRNA, the RNP binds to a predetermined site in a tumor genome (i.e., a site in the fusion), cuts the tumor genome, and promotes integration of the expression cassette there. The expression cassette includes an exogenous coding sequence encoding a gRNA complimentary to a sequence of a repetitive element in a human genome. Once integrated into the tumor genome, the exogenous coding sequence is expressed as gRNA in the tumor cell of a subject.

In other aspects of the invention, the invention provides methods, systems and compositions for inducing expression of gRNAs within a cell. The invention relies on a first Cas-related gene editing system to target a first site within a genome. The first site may be specific to a specific cell type, such as a fusion found only in a tumor genome. The Cas-related gene editing system includes a gRNA complimentary to at least a portion of the first site in the cell genome. The invention uses Cas-related endonucleases to induce expression of gRNAs that target at least one other site within the genome of the cell. The Cas-related gene editing system may also include an expression vector (e.g., an expression cassette) comprising nucleic acid encoding a gRNA complimentary to a second site in the cell. The Cas-related gene editing system targets, via the gRNAs, the first site in the cell genome and causes insertion of the nucleic acid encoding the gRNA complimentary to a second site. The second site gRNA is expressed within the cell and targets the second site with the associated Cas endonuclease and cleaves the nucleic acid at the second site. Without being bound by theory, methods and systems of the invention may induce expression of a plurality of gRNAs within the same cell. Each of the plurality of gRNAs may target a different site within the same cell. Thus, a plurality of sites may be targeted by the present invention. Methods and compositions of the invention are useful for inducing a cell to express CRISPR-like systems within the same cell that target multiple sites.

Methods and compositions of the disclosure are useful for treating a patient affected by a cancer, any proliferative disorder, or any other disease/disorder that results in a unique sequence in the genome. Methods and compositions of the disclosure may be used for treatment of any cancer such as melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer. Methods and compositions of the disclosure may be used for any virus that inserts itself into the human genome such as human immunodeficiency virus (HIV), human papilloma virus (HPV), and herpes simplex virus (HSV).

### BRIEF DESCRIPTION OF THE DRAWINGS

FIG. 1 diagrams a method for treating a tumor cell.

FIG. 2 diagrams a method for identifying fusions in a tumor genome.

FIG. 3 illustrates a gene editing system specifically targeting a tumor fusion to cause expression of a gRNA sequence in a tumor cell.

FIG. 4 illustrates an embodiment of a Cas-related gene editing system.

FIG. 5 diagrams an exemplary method for treating cancer in a subject using a Cas-related gene editing system that targets a tumor fusion and inserts a gRNA sequence specific to a known repeat element in the human genome to cause a tumor cell to express the gRNA and cleave the sequence of the known repeat element in the tumor cell.

FIG. 6 illustrates a CRISPR-Cas system expressed in a tumor cell.

### DETAILED DESCRIPTION

The disclosure provides methods and compositions that enable cells to express CRISPR-like systems. Particularly, the disclosure provides compositions and methods that enable cancer cells to express Cas-related endonucleases and gRNAs that target and cleave known repetitive elements within the human genome within the cancer cell. Compositions and methods of the disclosure use RNA-guided Cas endonucleases to target DNA fusions present only in cancer cells for the expression of Cas or Cas-related endonucleases and guide RNAs that target known repetitive elements in the human genome. Compositions and methods of the disclosure are useful to induce expression of Cas-related endonucleases and guide RNAs (gRNAs) complimentary to repetitive elements within the human genome (e.g., Alu and LI) exclusively in tumor cells. The expression of the exogenous CRISPR-like system in the tumor cell results in cleavage of the repetitive elements within the tumor DNA. Thus, the invention achieves specificity of action against cancer cells (via cancer-specific fusions) while targeting multiple known sequences for massive destruction of DNA—which leads to cell death.

The gene editing systems of the invention include Cas-related gene editing systems that include Cas-related endonucleases, associated gRNAs complimentary to a first target site in a cell (e.g., a fusion site), and nucleic acids encoding gRNAs complimentary to a predetermined second site (e.g., a known repetitive element within the human genome) within the same cell. Thus, the systems of the invention induce expression of CRISPR-like systems in target cells.

Clustered regularly interspaced short palindromic repeats (CRISPR) were originally found in bacterial genomes under common control with various CRISPR-associated (Cas) proteins. Cas protein 9 (Cas9) has since proven to be an RNA-guided endonuclease useful as a gene editing system when complexed with guide RNA. Cas9 is one Cas endonuclease and other, similar nucleases are known. Natively, the guide RNA included two short single-stranded RNAs, the CRISPR RNA (crRNA) that binds to the target in the target genetic material, and the trans-activating RNA (tracrRNA) that must also be present, although those two RNAs are commonly provided as a single, fused RNA sometimes called a single guide RNA (sgRNA). As used herein, guide RNA (gRNA) refers to either format. Cas9 and gRNA form a ribonucleoprotein (RNP) complex and bind to genomic DNA. The Cas9-gRNA complex scans the genome to identify a protospacer adjacent motif (PAM) and then a genomic DNA sequence adjacent to PAM that matches the gRNA sequence to cleave it. This scanning process depends on three-dimensional gRNA-dependent and gRNA-independent interactions of the Cas9-gRNA complex to DNA. The gRNA-dependent interaction is derived from the base-paring between a gRNA and genomic DNA. In contrast, the gRNA-independent interactions take place between genomic DNA and the amino acid residues of Cas9, including the PAM recognition. By virtue of the sequence of the gRNA, a Cas RNP cleaves target genetic material in a specific and controllable manner. Sequence-specific cleavage is useful for genome editing by, for example, providing a segment of DNA to be spliced in at the cleavage site by homology-directed repair.

To induce expression of gRNA in a specific cell, a CRISPR-associated (Cas) system can be delivered, along with an expression cassette for at least a gRNA, into a subject. The guide RNAs are designed and synthesized with predetermined targeting sequences and are thus unique reagents having a specific function. In Cas systems, the guide RNAs have sequences unique to a particular target site. The Cas system targets a predetermined site (a first site) in a tumor genome and provides for the insertion of a coding sequence at that site in the tumor genome. The coding sequence preferably encodes a gRNA complementary to a second site within the tumor genome. Preferably, the second site is one or more repetitive sequences found in the human genome. Once the coding sequence is integrated at the predetermined site of the tumor genome (which may be, for example, a genomic safe harbor), the coding sequence, i.e., the gRNA complementary to a second site within the tumor genome (for example, a repetitive sequence in the human genome), is then expressed in tumor cells. Because healthy, non-tumor cells do not have matching sites in their genomes, only the tumor cells then express the inserted gRNA, whereby the tumor cells can be destroyed with the associated Cas-associated endonuclease. Thus, by inducing human cells to express a CRISPR-like system, human cells can also benefit from sequence-specific cleavage. Sequence-specific cleavage is useful for inducing cell death by, for example, targeting gRNAs to known repetitive elements in the human genome for cleavage. However, because known repetitive elements are found in both diseased (e.g., tumor cells) and normal cells, the induced CRISPR-Cas system must be specific to tumor cells.

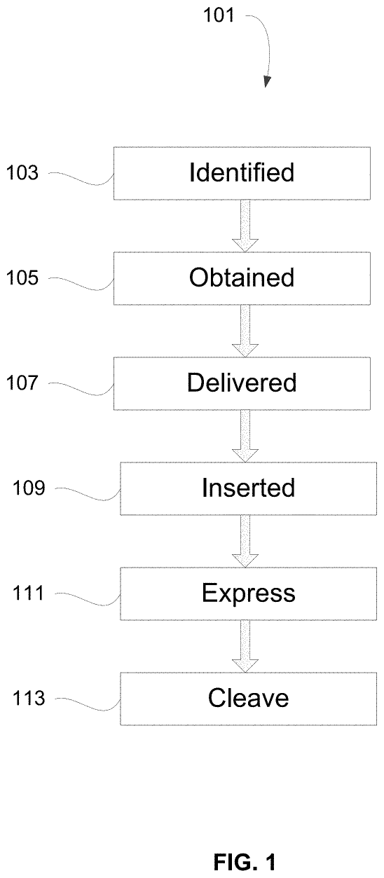

FIG. 1 diagrams a method 101 of treating a tumor cell. In the method 101, one or more fusion(s) found only in tumor genomes are identified 103. Nucleic acid sequences encoding Cas-associated endonucleases and nucleic acid encoding gRNAs (i.e., nucleic acid encoding CRISPR-like systems for expression in tumor cells) are obtained 105. The nucleic acid sequences may be packaged as a recombinant DNA molecule. The naked nucleic acid, or the recombinant DNA molecules, may include an expression control sequence. The nucleic acid encoding the CRISPR-Cas system is delivered 107 to a tumor cell by targeting one or more fusions identified in the tumor genome. The naked nucleic acid or the recombinant DNA molecules may be delivered 107 via a Cas-related gene editing system that targets the one or more fusion(s). The gRNAs of the Cas-related gene editing system may target at least a portion of the one or more fusion(s) and insert 109 at least the nucleic acid encoding gRNAs. The nucleic acid encoding gRNAs is complimentary to a second site in the same cell. Here, the second site it at least one known repetitive sequence. The guide RNAs of the Cas-related gene editing system are designed and synthesized with predetermined targeting sequences and are thus unique reagents having a specific function. In Cas systems, the guide RNAs have sequences unique to a particular target site. Here, the particular site is a first site, or a fusion sequence in a tumor genome.

The Cas-related gene editing system includes the nucleic acid encoding gRNAs that are complimentary to at least one known repetitive sequence in the human genome. Because healthy, non-tumor cells do not have matching sites in their genomes, only the tumor cells express 111 the gRNAs system. The nucleic acid encoding the guide RNAs are designed and synthesized with predetermined targeting sequences and are also unique reagents having a specific function. The expressed gRNAs in tumor cells target a predetermined sequence of or within a known repetitive element of the human genome. The CRISPR-like system expressed 111 in the tumor cell cleaves 113 the tumor material at the repetitive sequence. Known repetitive elements occur frequently throughout the genome, providing multiple sites for cleaving and thus destruction of tumor cell DNA. The tumor cells may then be destroyed by programmed cell death, e.g., apoptosis, or may die simply by destruction of their DNA.

Methods and compositions of the invention are useful for treating any proliferative disease or disorder, such as cancer. The disclosure provides Cas-related strategies, as well as methods and compositions that induce expression of CRISPR-like systems in tumor-specific cells, or any cell in need of treatment, thereby creating an immune system within a human cell comparable to bacterial cell immune systems, that is capable of destroying the cell from the inside out.

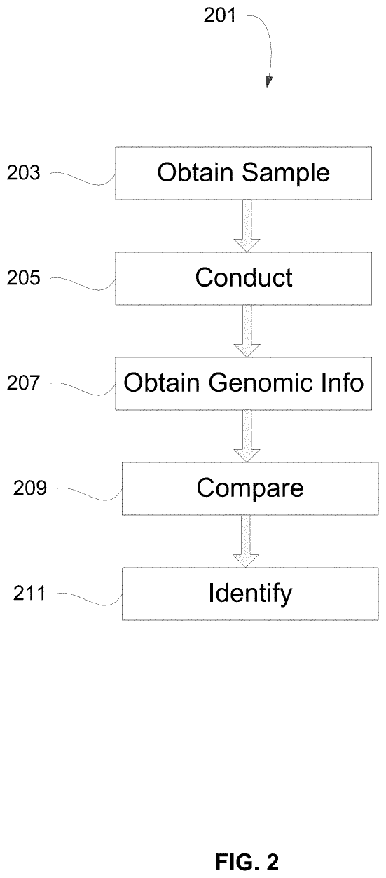

FIG. 2 diagrams a method 201 of identifying fusions in tumor-specific genomic material of a subject. In the method 201, a sample is obtained 203 from a subject. Patient samples are obtained 201 that preferably include both tumor DNA and healthy, non-tumor DNA. Samples may be obtained from any suitable germline or somatic sources (e.g., buccal or blood). Tumor cells may be obtained by tumor biopsy or circulating tumor cells may be isolated using methods known in the art.

An assay is conducted 205 on the sample and genomic information is obtained 207. For example, tumor and matched-normal DNA may be sequenced (e.g., on an Illumina sequencing instrument) to obtain tumor and matched-normal sequences. By such a manner, the genomic information of a non-tumor sample is compared 209 to genomic information of the tumor cell, fusions are identified 211 in the latter. For example, the whole-genome sequence of tumor and matched-normal DNA may be compared 209. Tumor-specific genomic material (e.g., fusions) specific to tumor cells is identified 211 from the comparison. Comparing 209 may include comparing tumor sequences to matched-normal sequences (e.g., by alignment of assembled sequences from an NGS instrument run). Tumor-specific genomic material may include fusions specific to a tumor cell. Thus, a distinguishing feature of the identified 211 fusions is that it is not also found in “matched normal” sequences from healthy, non-tumor cells. Methods (e.g., 101) of the invention use fusions identified 211 by the method 201 as a target for a targeting moiety (e.g., an antibody, aptamer, ligand, nucleic acid (e.g., an expression control sequence), peptide, protein, receptor, or any other molecule that facilitates binding to one or more fusion(s) on a tumor cell) to deliver to a tumor cell, nucleic acid encoding Cas-related endonucleases and gRNAs complimentary to sequences of known repetitive sequences in the human genome to cause expression of Cas-related endonucleases and the gRNAs in tumor cells only. Preferably, methods 101 of the invention use fusions identified 211 by the method 201 as a target for a Cas-related gene editing systems of the invention to cause insertion 109 of a nucleic acid encoding gRNA complimentary to at least one known repetitive sequence in the human genome into the fusion sequence, and thus the expression 111 of the gRNA in the tumor cell only.

Sequencing may be by any method known in the art. See, generally, Quail, et al., 2012, A tale of three next generation sequencing platforms: comparison of Ion Torrent, Pacific Biosciences and Illumina MiSeq sequencers, BMC Genomics 13:341. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing.

An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Another example of a DNA sequencing technique that can be used is the sequencing-by-ligation technology offered under the tradename SOLiD by Applied Biosystems from Life Technologies Corporation (Carlsbad, Calif.). In SOLiD sequencing, genomic DNA is sheared into fragments, and adaptors are attached to generate a fragment library. Clonal bead populations are prepared in microreactors containing beads, primers, template, and PCR components. Following PCR, the templates are denatured and enriched and the sequence is determined by a process that includes sequential hybridization and ligation of fluorescently labeled oligonucleotides.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, each incorporated by reference. DNA is fragmented and given amplification and sequencing adapter oligos. The fragments can be attached to a surface. Addition of one or more nucleotides releases a proton (H+), which signal is detected and recorded in a sequencing instrument.

Other examples of a sequencing technology that can be used include the single molecule, real-time (SMRT) technology of Pacific Biosciences (Menlo Park, Calif.) and nanopore sequencing as described in Soni and Meller, 2007 Clin Chem 53:1996-2001. Such sequencing methods are useful when obtaining large fragments of DNA from a reference or test sample, such as in the methods described in U.S. Pub. 2018/0355408, the contents of which are incorporated by reference herein.

Sequencing tumor DNA provides tumor sequences that may be analyzed to identify fusions that appear exclusively in tumor genomes and do not appear in a genome from a healthy, non-tumor cell from the same subject.

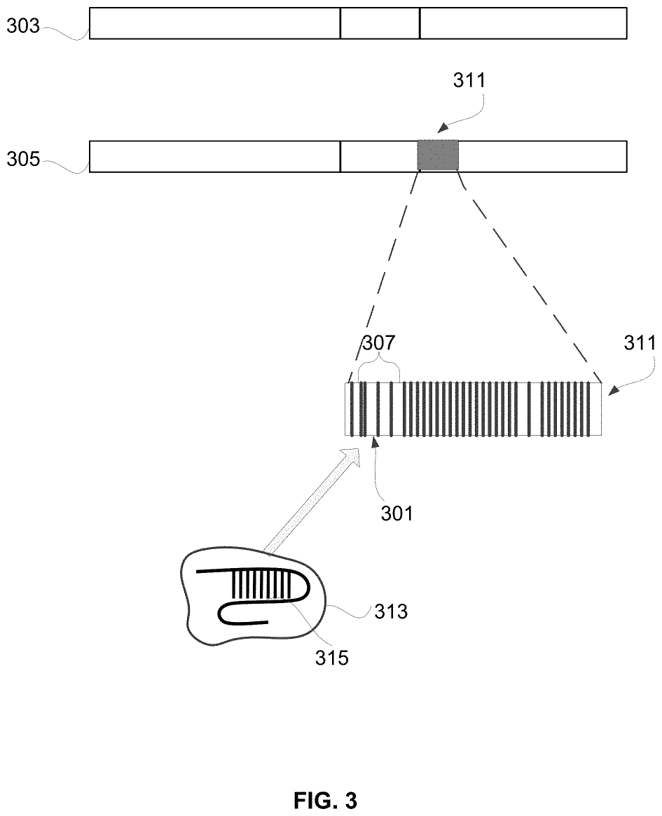

FIG. 3 illustrates the analysis of tumor sequence 305 to identify tumor-specific genomic material 311 (e.g., a fusion). In the depicted embodiment, tumor sequence 305 is aligned to matched normal sequences 303 to determine any differences. Where the tumor sequences 305 include tumor-specific genomic material 311 that are not also present in the matched normal sequences 303, that tumor-specific genomic material 311 provides a target for cleavage by a gene editing system and subsequent integration (e.g., by homology directed repair) of an expression cassette bearing, e.g., exogenous coding sequence.

More particularly, in the depicted embodiment, a segment 307 of the tumor-specific genomic material 311 (e.g., DNA) is shown. The gene editing system is designed to recognize that segment and cleave the tumor DNA at a target 301. Because the matched normal DNA does not include the tumor-specific genomic material 311, a healthy, non-tumor genome does not include a corresponding segment 307 that can be recognized by the gene editing system 313 and thus the gene editing system 313 has no relevant effect on healthy, non-tumor cells. A distinguishing feature of the segment 307 is that the segment 307 includes features that satisfy the targeting requirement of the gene editing system 313. Thus, a distinguishing feature of the tumor-specific material 311 is that it is not also found in “matched normal” sequences from healthy, non-tumor cells. The segment 307 within the tumor material 311 includes matches for the targeting sequence of gene editing system 313. Where, for example, the gene editing system 313 uses a Cas endonuclease, the segments 307 are those locations that include a suitable PAM adjacent to a suitably specific approximately 20 base target.

Using this information, one of skill in the art can prepare or obtain gene editing systems useful to insert a copy of a nucleotide sequence encoding a gRNA at the target 301. For example, one may access the sequence of the tumor-specific genomic material from the method 201 of comparing 209 germline DNA to tumor DNA to search for and identify targets suitable for insertion and editing with a Cas-related gene editing system 313.

In a preferred embodiment, the Cas-related gene editing system uses Cas endonuclease and guide RNA. For example, the Cas endonuclease may be Cas9 from Streptococcus pyogenes (spCas9). The Cas endonuclease may be complexed with a guide RNA 315 as a ribonucleoprotein (RNP). One of skill in the art may design the gRNA 315 to have a 20-base targeting sequence complementary to the segment 307 of the tumor-specific genomic material 311. Alternatively, the gRNA 315 may have a 20-base targeting sequence complementary to a target within a few hundred or thousand bases of the segment 307.

The target may be a sequence describable as 5′-20 bases-protospacer adjacent motif (PAM)-3′, where the PAM depends on Cas endonuclease (e.g., NGG for Cas9). To insert an exogenous gRNA, two Cas RNPs may be used along with a pair of guide RNAs 309 to flank the target 301. The RNPs bind to their cognate targets in the tumor-specific DNA 305 and introduce double stranded breaks. The nucleotide sequence encoding the gRNA being inserted may have ends that are homologous to sequences flanking the target 301 to induce the cell's endogenous homology-directed repair response, to repair the genome by inserting the exogenous DNA segment. See How, 2019, Inserting DNA with CRISPR, Science 365(6448):25 and Strecker, 2019, RNA-guided DNA insertion with CRISPR-associated transposases, Science 365(6448):48, both incorporated herein by reference. Thus, in the depicted embodiment, the sequence encoding the gRNA is inserted into the tumor-specific DNA 311 only using a CRISPR/Cas nuclease system. The method 101 may be performed with any suitable gene editing system. A Cas nuclease system uniquely corresponds to intended targets, such as a predetermined site in the fusion. The predetermined site may be near the promoter region of a tumor specific gene. In some embodiments, the target site may be within an open reading frame (ORF) in the tumor-specific genomic material, and genome editing can integrate the exogenous coding sequence, in-frame, within the ORF. Insertion of the coding sequence into the ORF causes expression of the gRNA within the tumor cell. Gene editing systems can be designed and synthesized or ordered by making reference to the predetermined site in the tumor-specific genomic material. Alternatively, nucleotide sequence of a gRNA (e.g., a gRNA complimentary to a known repetitive element in the human genome) and a suitable promoter can be expressed in a safe harbor, using Cas systems described herein.

Embodiments of the invention use any suitable gene editing system such as, for example, CRISPR systems, transcription activator like effector nucleases (TALENs), zinc finger nucleases, or meganucleases. In any embodiment discussed herein, gene editing system may be taken to refer to compositions that include an active form of the protein or that include a nucleic acid encoding the gene editing system. Thus, a CRISPR system can include a Cas-endonuclease complexed with a guide RNA as an RNP, or a nucleic acid encoding those elements, such as on a plasmid or other expression cassette. Preferred embodiments of the invention use a CRISPR-associated (Cas) endonuclease. The gene editing system includes a protein (i.e., a Cas endonuclease) that is complexed with target-specific gRNA, thus forming a complex that targets the Cas endonuclease to a specific sequence in the tumor-specific genomic material (i.e., the identified fusion). Any suitable Cas endonuclease or homolog thereof may be used. A Cas endonuclease may be Cas9 (e.g., spCas9), Cpf1 (aka Cas12a), C2c2, Cas13, Cas13a, Cas13b, e.g., PsmCas13b, LbaCas13a, LwaCas13a, AsCas12a, PfAgo, NgAgo, CasX, CasY, others, modified variants thereof, and similar proteins or macromolecular complexes.

The Cas endonuclease of the gene editing system may also be used by the gRNAs expressed in the tumor cell by the gene editing system. In some embodiments, the gene editing system also includes nucleic acid encoding a Cas-related endonuclease to associate with the gRNAs once expressed in the target cell.

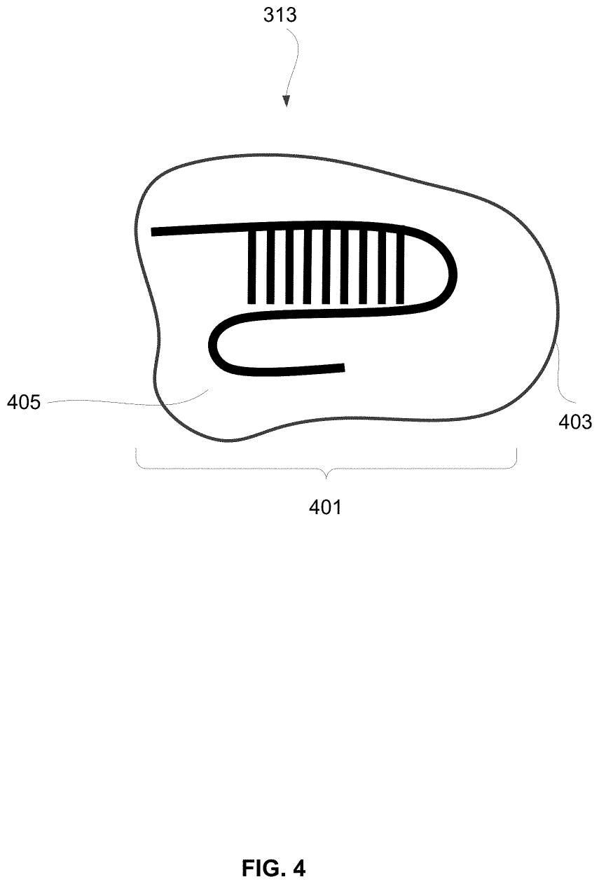

FIG. 4 shows an embodiment of Cas-related gene editing system 313. The depicted embodiment includes a Cas endonuclease 403 and a guide RNA 405 (i.e., gRNA). The gRNA 405 includes a targeting sequence of approximately 20 bases complementary or nearly complementary to a target in tumor-specific genomic material of a subject. The Cas endonuclease 403 and gRNA 405 are complexed together into a ribonucleoprotein (RNP) 401. The CRISPR/Cas system 313 in a composition or method of the disclosure may include at least one Cas endonuclease 403 (or a nucleic acid encoding the Cas endonuclease).

The host bacteria capture small DNA fragments (˜20 bp) from invading viruses and insert those sequences (protospacers) into their own genome to form a CRISPR. CRISPR regions are transcribed as pre-CRISPR RNA(pre-crRNA) and processed to give rise to target-specific crRNA. Invariable target-independent trans-activating crRNA (tracrRNA) is also transcribed from the locus and contributes to the processing of precrRNA. The crRNA and tracrRNA have been shown to be combinable into a single guide RNA (gRNA). As used herein, “guide RNA” or gRNA refers to either format. The gRNA forms a RNP with Cas9, and the RNP cleaves a target that includes a portion complementary to the guide sequence in the gRNA, as well as a sequence known as protospacer adjacent motif (PAM). The RNPs are programmed to target a specific viral nucleic acid by providing a gRNA having a ˜20-bp guide sequence that is complementary or substantially complementary to a target in viral nucleic acid. The targetable sequences include, but are not limited to: 5′-X 20NGG-3″ or 5′-X 20NAG-3″; where X 20 corresponds to the 20-bp crRNA sequence and NGG and NAG are PAMs. Sequences with lengths other than 20 bp and PAMs other than NGG and NAG are known and are included within the scope of the invention.

Any of the CRISPR/Cas system compositions and methods of the disclosure may be included in any suitable format, and including any of protein, messenger RNA, DNA, RNP, or a combination thereof. For example, delivery of RNPs into cells may be by electroporation, chemical poration, or via liposomal mediated delivery. The nucleotide sequence encoding a cell surface protein may be included as a segment of DNA that also includes one or more of a promoter, a fluorescent protein, an SV40 sequence, and a poly(A) sequence. The nucleotide sequence encoding a cell surface protein may be included in an expression cassette along with one or more of a promoter, a fluorescent protein, an SV40 sequence, and a poly(A) sequence. The sequence (e.g., expression cassette) and/or the gene editing system may be delivered as a plasmid or other similar vector. The components of the systems may be delivered in a DNA-sense (e.g., as a plasmid or in a viral vector) for transcription and translation into active proteins in the tumor cells. In some embodiments, a gene editing system 313 is delivered as nucleic acid, e.g., the Cas endonuclease, and is packaged with a nucleotide sequence encoding a guide RNA complimentary to a second site (e.g., a sequence of a known repetitive element) using one or more lentiviral or adeno-associated virus (AAV) vector.

The gene editing system may be delivered in a protein, RNP, DNA, or mRNA format dependent on a desired persistence or stability in the tumor cells. The gene editing system may include an endonuclease designed to introduce a gRNA into a target site of the fusion. Target sites may include a gene locus of a tumor cell gene, such as a fusion, a predetermined site in tumor-specific genomic material, such as a tumor-specific locus of a tumor-specific gene of a subject or a genomic safe harbor (e.g., a safe harbor such as AAVS1, CCR5, or ROSA26.). The gene editing system may be included as DNA that is transcribed after the composition is introduced into subject as mRNA or as a protein or RNP. Regardless of format, a suitable packaging vector or particle may be used.

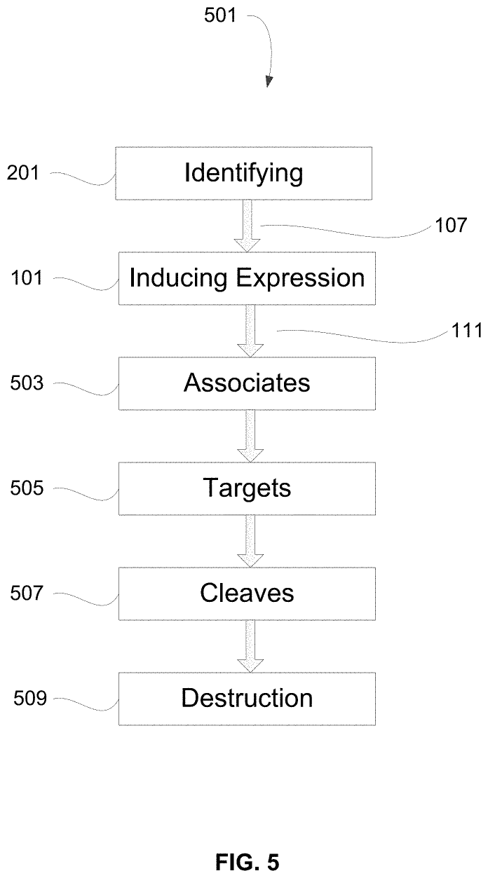

FIG. 5 depicts an exemplary method 501 of inducing expression of a gRNA in a cell using a gene editing system 313 of the present invention. In the method 501, the method of identifying a fusion specific to a tumor genome of a subject 201 is performed. Upon identification of the fusion, the method 101 is performed. Once the nucleic acid encoding the gRNA is inserted into the fusion tumor cells express 211 the gRNA, the gRNA associates 503 with the Cas-related endonuclease in the tumor cell. The gRNA is specific to a second site, such as sequence of a known repetitive element in the human genome. The gRNA targets 505 the second site and the Cas-related endonuclease cleaves 507 the tumor genome at the second target site. When the second site is a repetitive sequence, the Cas-related endonuclease cleaves 507 a plurality of site having the same sequence. Thus, the cleavage of a plurality of second sites in the tumor genome results in the destruction 509 of the tumor cell.

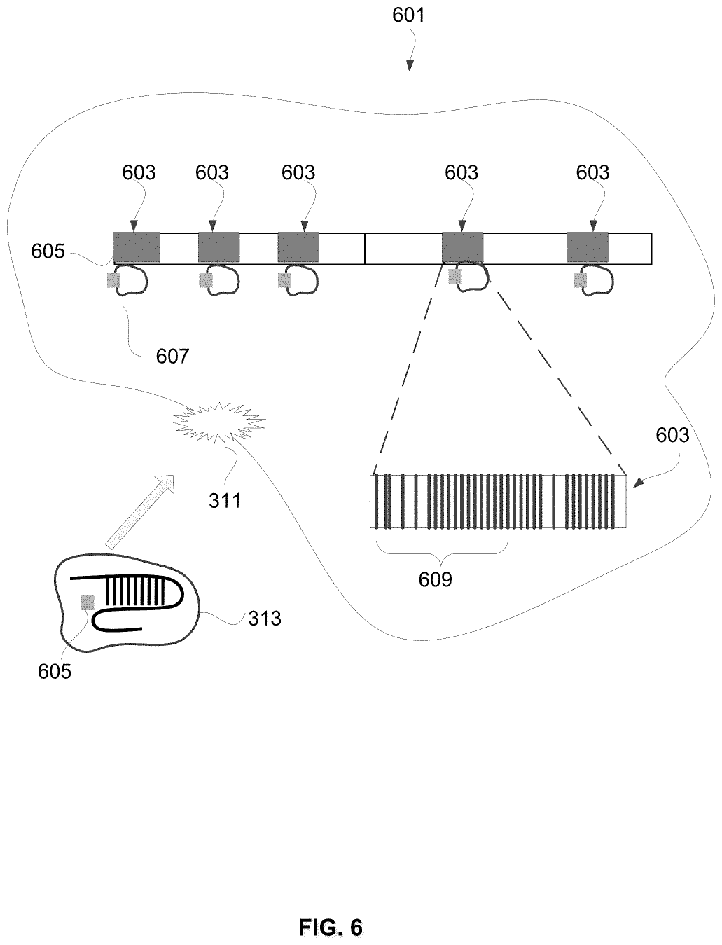

FIG. 6 depicts an exemplary tumor cell 601 having known repetitive elements 603 of the human genome throughout. A Cas-related endonuclease gene editing system 313 having nucleic acid sequences encoding gRNAs 605 are delivered into the tumor cell 601 by targeting one or more fusions 311 identified 211 in the tumor genome. The nucleic acid 605 may include an expression control sequence (not shown). In some embodiments, the nucleic acid 605 may be part of a recombinant DNA molecule (not shown), such as a plasmid or a vector.

Once inside the cell 601 and expressed 111, the activated CRISPR-like system 607 is designed to recognize segments 609 of known repetitive elements 603 and cleave the tumor DNA 305 at those segments. Because at least the gRNAs 605 are only delivered and expressed in tumor cells 601, the repetitive sequences in normal cells will not be targeted by those gRNAs 605, and thus the expressed CRISPR-like system 607 has no relevant effect on healthy, non-tumor cells. A distinguishing feature of the repetitive segment 609 is that the segment 609 includes features that satisfy the targeting requirement of the induced CRISPR-like system 607. The segment 609 within the tumor material includes matches for the targeting sequence of the gRNA 605. For example, the induced CRISPR-like system 607 uses a Cas endonuclease delivered to the tumor cell via the Cas-related gene editing system 313, the repetitive segments 609 targeted may include locations that include a suitable PAM adjacent to a suitably specific approximately 20 base target of a repetitive sequence 603. There will, however, be instances in the tumor genome where the target repetitive sequence 603 is not adjacent a suitable PAM sequence. In such instances, the induced CRISPR-Cas system will skip that target repetitive sequence 603. However, because there are many (e.g., 100 s, 1,000 s, 1,000,000 s) of the same target repetitive sequences 603 within the tumor cell genome, many other sites of the target repetitive sequences 603 will be adjacent a suitable PAM sequence. Thus, the induced CRISPR-like system will continue to cleave the tumor material at those sites adjacent a suitable PAM sequence.

Any repetitive element that exists in numerous copies within a genome may be used as a target for the expressed gRNA. That is, the nucleic acid sequences encoding the gRNAs of the present invention are such that when expressed in a tumor cell, the gRNAs are complimentary to a sequence of a repetitive element. Repetitive elements 603 in the human genome include satellite DNA, tandem repeats, transposons, interspersed retrotransposons (e.g., long interspersed repetitive elements (LINEs), and short interspersed repetitive elements (SINEs)). Repetitive elements may differ in their position in the genome, for purposes of the present invention because their sequences are known and they occur frequently, their location in the tumor DNA does not matter. That is, the gRNAs 605 are complimentary to segments 609 of repetitive elements 603 and will hybridize to those segments regardless of their location in the genome. Preferably, the segment of the repetitive element is a 5′-20 bases-protospacer adjacent motif (PAM)-3′.

Repetitive elements 603 include, for example SINEs present in hundreds of thousands of copies scattered across the human genome. Repetetive elements may be from 7 to 3,000 base pairs in length. Thus, in some embodiments of the invention, gRNAs target segments of repetitive element sequences. Preferably, those segments are 5′-20 bases-protospacer adjacent motifs (PAM)-3′ and the gRNAs are complimentary thereto. SINEs include Alu sequences, comprising a 282 consensus sequence, typically followed by an A-rich region and flanked by direct repeat sequence representing the duplicated insertion site. Alus are repeated on average, every 3,000 base pairs in the human genome, and thus are targets for gRNA expressed in tumor cells via systems and methods of the present invention. Repetitive elements according to this invention are described in Asmit AFA, Hubley R & Green, P. RepeatMasker Open-4.0. 2013-2019 http://www.repeatmasker.org; Piégu, Benoît, et al. A survey of transposable element classification systems—a call for a fundamental update to meet the challenge of their diversity and complexity. Molecular phylogenetics and evolution 86 (2015): 90-109; Kapitonov, Vladimir V., and Jerzy Jurka. A universal classification of eukaryotic transposable elements implemented in Repbase. Nature Reviews Genetics 9.5 (2008): 411-412; Wicker, Thomas, et al. A unified classification system for eukaryotic transposable elements. Nature Reviews Genetics 8.12 (2007): 973-982; and Curcio, M. Joan, and Keith M. Derbyshire. The outs and ins of transposition: from mu to kangaroo. Nature Reviews Molecular Cell Biology 4.11 (2003): 865-877, each incorporated herein by reference in their entirety.

In a preferred embodiment, the CRISPR-like system is a gene editing system expressed by tumor cells themselves and includes Cas endonuclease and guide RNA. For example, the Cas endonuclease may be Cas9 from Streptococcus pyogenes (spCas9). One of skill in the art may design the nucleic acid encoding the gRNA to have a 20-base targeting sequence complementary to the segment of the repetitive element when expressed in the tumor cell. Alternatively, the gRNA, when expressed in the tumor cell, may have a 20-base targeting sequence complementary to a target within a few hundred or thousand bases of the segment of the repetitive element.

The repetitive element may be a sequence describable as 5′-20 bases-protospacer adjacent motif (PAM)-3′, where the PAM depends on Cas endonuclease (e.g., NGG for Cas9). To cleave segments of the tumor DNA, two Cas-related endonucleases may be used along with a pair of guide RNAs to flank a segment of a repetitive element sequence. The Cas-gRNA form a complex and bind to their cognate targets in the tumor-specific DNA and introduce double stranded breaks, causing the tumor DNA to fragment. In an aspect of the invention, a deletion is caused by positioning two double strand breaks proximate to one another, thereby causing a fragment of the genome to be deleted. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11, Horvath et al., Science (2010) 327:167-170; Terns et al., Current Opinion in Microbiology (2011) 14:321-327; Bhaya et al. Annu Rev Genet (2011) 45:273-297; Wiedenheft et al. Nature (2012) 482:331-338); Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918), each incorporated by reference. In the depicted embodiment, the expressed system 607 is a CRISPR/Cas nuclease system. The method 101 may be performed with any suitable Cas (as described above) and guide RNA that can be expressed in a human cell by methods of the invention. The gRNAs expressed by a human cell uniquely correspond to intended targets, such as a predetermined sequence of a repetitive element within in the human genome. Nucleic acid encoding Cas-related endonucleases and gRNAs of the present invention can be designed and synthesized or ordered by making reference to the predetermined site within the repetitive element in the human genome.

The sequences encoding the gRNA and/or the Cas-related endonuclease may be delivered as a plasmid or other similar vector for insertion and expression in the tumor cell. The gRNA and/or the Cas-related endonuclease components may be delivered in a DNA-sense (e.g., as a plasmid or in a viral vector) for transcription and translation into active proteins in the tumor cells. An expression vector is a specialized vector that contains the necessary regulatory regions needed for expression of the components of interest in a tumor cell. In some embodiments the components are operably linked to another sequence in the vector. The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and expression control elements (e.g. promoters, enhancers, and termination elements) in an expression vector.

Many viral vectors or virus-associated vectors are known in the art. Such vectors can be used as carriers of a nucleic acid construct into the cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, such as an Eptsein Barr virus (EPV or EBV) vector. The inserted material of the vectors (i.e., the components of the CRISPR-Cas systems) described herein may be operatively linked to an expression control sequence when the expression control sequence controls and regulates the transcription and translation of that nucleotide sequence. In some examples, transcription of an inserted material is under the control of a promoter sequence (or other transcriptional regulatory sequence) which controls the expression of the recombinant nucleic acid.

In some embodiments, the expression vector is a lentiviral vector. Lentiviral vectors may include a eukaryotic promoter. The promoter can be any inducible promoter, including synthetic promoters. In addition, the lentiviral vectors used herein can further comprise a selectable marker, which can comprise a promoter and a coding sequence for the gRNAs and the Cas-related endonucleases. Nucleotide sequences encoding selectable markers are well known in the art.

In some embodiments the viral vector is an adeno-associated virus (AAV) vector. AAV can infect both dividing and non-dividing cells and may incorporate its genome into that of the host cell. One suitable viral vector uses recombinant adeno-associated virus (rAAV).

In certain embodiments, as an alternative to a Cas-related gene editing system, the nucleic acid or the vector may be linked to a targeting moiety that facilitates delivery of the nucleic acid encoding a gRNA and a Cas-related endonuclease to a target cell, i.e., a tumor cell. The targeting moiety may bind to a target, such as a fusion, on or in a tumor cell. Methods of making and attaching targeting moieties are well-known in the art. Targeting moieties may include proteins (mainly antibodies and their fragments), peptides, nucleic acids (aptamers), small molecules, or others (vitamins or carbohydrates). Embodiments of the invention include making or obtaining individual preselected peptides or RNAs that target specific proteins (i.e., one or more fusion(s)) to be expressed from recombinant DNA molecules (e.g., plasmids or vectors) encoding nucleic acid of gRNAs and Cas-related endonucleases of the present invention. Exemplary peptides and methods of making targeting moieties include Noncovalent Attachment of Chemical Moieties to siRNAs Using Peptide Nucleic Acid as a Complementary Linker ACS Appl Bio Mater. 2018 Sep. 17; 1(3): 643-651, incorporated herein by reference in its entirety. In some embodiments, a recombinant cell containing an inducible promoter is used and exposed to a regulatory agent or stimulus by externally applying the agent or stimulus to the cell or organism by exposure to the appropriate environmental condition or the operative pathogen. Inducible promoters initiate transcription only in the presence of a regulatory agent or stimulus. Examples of inducible promoters include the tetracycline response element and promoters derived from the beta-interferon gene, heat shock gene, metallothionein gene or any obtainable from steroid hormone-responsive genes. Tissue specific expression has been well characterized in the field of gene expression and tissue specific and inducible promoters are well known in the art. These promoters are used to regulate the expression of the foreign gene after it has been introduced into the target cell. In certain embodiments, a cell-type specific promoter or a tissue-specific promoter is used. A cell-type specific promoter may include a cell-type specific promoter, which regulates expression of a selected nucleic acid primarily in one cell type, and not in other cells, by virtue of annealing to cell-specific sequence, such as a fusion found in a tumor genome. Methods of making and delivering plasmids and vectors are well known in the art, for example Naso, M., et al., Adeno-Associated Virus (AAV) as a Vector for Gene TherapyAdeno-Associated Virus (AAV) as a Vector for Gene TherapyBioDrugs. 2017; 31(4): 317-334; and Rmamoorth, M., et al., Non Viral Vectors in Gene Therapy—An Overview, J Clin Diagn Res. 2015 January; 9(1): GE01-GE06, each incorporated by reference herein in their entirety.

Methods of the invention also include inhibiting tumor growth or metastasis of cancer in a subject by administering to the subject a therapeutically effective amount of the compositions disclosed herein. A therapeutically effective amount of the compositions disclosed herein is an amount sufficient to inhibit growth, replication or metastasis of cancer cells, or to inhibit a sign or a symptom of the cancer. The therapeutically effective amount may depend on disease severity, the type of disease, or the subject's general health.

Any suitable delivery system may be used to deliver the Cas-related gene editing systems of the present invention. Delivery methods are described in detail in Wilbie, 2019, Delivery aspects of CRISPR/Cas for in vivo genome editing, Acc Chem Res 18; 52(6):1555-1564, incorporated by reference. Non-viral delivery of the systems of the present invention can be used. For example, liposome(s) may be used to deliver a gene editing system or nucleic acid encoding the gene editing system along with an expression cassette for an exogenous coding sequence. Any nucleic acid delivered may be as a plasmid that may also include a segment that encodes a gRNA. Where the liposome packages nucleic acids, the nucleic acids may include one or any combination of a plasmid, a guide RNA, and the expression cassette. Compositions may be packaged in a plurality of the liposomes. Each of the plurality of liposomes may envelope one or more of an expression cassette and/or the gene editing system (e.g., in protein or plasmid format). Delivery of the liposomes to tumor cells in a subject causes those cells to express the gRNA in a stable manner.

Other embodiments use lipid nanoparticles such as solid lipid nanoparticles. A lipid nanoparticle (LNP) may include a gene editing system. LNPs may be about 100-200 nm in size and may optionally include a surface coating of a neutral polymer such as PEG to minimize protein binding and unwanted uptake. The nanoparticles are optionally carried by a carrier, such as water, an aqueous solution, suspension, or a gel. For example, LNPs may be included in a formulation that may include chemical enhancers, such as fatty acids, surfactants, esters, alcohols, polyalcohols, pyrrolidones, amines, amides, sulfoxides, terpenes, alkanes and phospholipids. LNPs may be suspended in a buffer. The buffer may include a penetration enhancing agent such as sodium lauryl sulfate (SLS). SLS is an anionic surfactant that enhances penetration into the skin by increasing the fluidity of epidermal lipids. Lipid nanoparticles may be delivered via a gel, such as a polyoxyethylene-polyoxypropylene block copolymer gel (optionally with SLS). Poloxamers are nonionic triblock copolymers composed of a central hydrophobic chain of poloxypropylene (poly(propylene oxide)) flanked by two hydrophilic chains of polyoxyethylene (poly(ethylene oxide)). Because the lengths of the polymer blocks can be customized, many different poloxamers exist having different properties. For the generic term “poloxamer”, these copolymers are commonly named with the letter “P” (for poloxamer) followed by three digits: the first two digits×100 give the approximate molecular mass of the polyoxypropylene core, and the last digit×10 gives the percentage polyoxyethylene content (e.g. P407=poloxamer with a polyoxypropylene molecular mass of 4,000 g/mol and a 70% polyoxyethylene content). LNPs may be freeze-dried (e.g., using dextrose (5% w/v) as a lyoprotectant), held in an aqueous suspension or in an emulsification, e.g., with lecithin, or encapsulated in LNPs using a self-assembly process. LNPs are prepared using ionizable lipid L319, distearoylphosphatidylcholine (DSPC), cholesterol and PEG-DMG at a molar ratio of 55:10:32.5:2.5 (L319:DSPC:cholesterol:PEG-DMG). The payload may be introduced at a total lipid to payload weight ratio of ˜10:1. A spontaneous vesicle formation process is used to prepare the LNPs. Payload is diluted to ˜1 mg/ml in 10 mmol/l citrate buffer, pH 4. The lipids are solubilized and mixed in the appropriate ratios in ethanol. Payload-LNP formulations may be stored at −80° C. See Maier, 2013, Biodegradable lipids enabling rapidly eliminating lipid nanoparticles for systemic delivery of RNAi therapeutics, Mol Ther 21(8):1570-1578, incorporated by reference. See, WO 2016/089433 A1, incorporated by reference herein.

Compositions of the disclosure may include a plurality of lipid nanoparticles having the nucleic acid encoding the gRNA and the gene editing system embedded therein. In one embodiment, a plurality of lipid nanoparticles comprises at least a solid lipid nanoparticle comprising a segment of DNA that encodes the gRNA; a second solid lipid nanoparticle that includes at least one Cas endonuclease complexed with a gRNA that targets the CRISPR/Cas system to a locus within a predetermined site in the tumor-specific genomic material (i.e., the fusion) of a subject.

In methods of treating cancer according to the disclosure, a therapeutically effective amount of a composition is administered to a subject. A therapeutic amount is an amount that is sufficient to cause a cancer cell to express a Cas-related endonucleases and gRNAs that form a complex in the cancer cell that targets segments of known repetitive elements in a human genome. Accordingly, methods of the disclosure include treating cancer in a subject by administering to the subject a therapeutically effective amount of the compositions disclosed herein.

In general, an effective dosage of any of the compositions of the present invention can readily be determined by a skilled person, having regard to typical factors such as the age, weight, sex and clinical history of the patient. A typical dosage could be, for example, 1-1,000 mg/kg, preferably 5-500 mg/kg per day, or less than about 5 mg/kg, for example administered once per day, every other day, every few days, once a week, once every two weeks, or once a month, or a limited number of times, such as just once, twice or three or more times. Methods of the invention include delivering an effective amount of the composition to the subject such that expression of gRNAs and Cas-related endonucleases occurs in a tumor cell, and the CRISPR-like system cleaves the tumor DNA at segments of the repetitive elements.

The disclosure also provides pharmaceutical compositions of the compositions described herein. Compositions may be formulated for delivery by any route of administration. For example, compositions may be formulated for oral, enteral, parenteral, subcutaneous, intravenous, or intramuscular administration.

Formulations may provide aqueous suspensions, oil suspensions, dispersible powders, or emulsions. The aqueous suspensions may contain one or more compounds in admixture with excipients suitable for the manufacture of aqueous suspensions. Oily suspensions may be formulated by suspending the compound in a suitable oil such as mineral oil, arachis oil, olive oil, or liquid paraffin. The oily suspensions may contain a thickening agent, for example beeswax, hard paraffin or cetyl alcohol. Dispersible powders and granules suitable for preparation of an aqueous suspension by the addition of water provide the compound in admixture with a dispersing or wetting agent, suspending agent and one or more preservatives. Suitable dispersing or wetting agents and suspending agents are exemplified, for example sweetening, flavoring and coloring agents, may also be present.

The compositions may also be in the form of oil-in-water emulsions. The oily phase may be a lipid, a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

Compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol. sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Compositions may be in a form suitable for oral use. For example, oral formulations may include tablets, troches, lozenges, fast-melts, aqueous or oily suspensions, dispersible powders or granules, emulsions, hard or soft capsules, syrups or elixirs. Formulations for oral use may also be presented as hard gelatin capsules in which the citrate, citric acid, or a prodrug, analog, or derivative of citrate or citric acid is mixed with an inert solid diluent, for example calcium carbonate, calcium phosphate or kaolin, or as soft gelatin capsules in which the compound is mixed with water or an oil medium, for example peanut oil, liquid paraffin or olive oil.

Pharmaceutical compositions of the disclosure may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Any of the compositions may be included in a kit. The kit may include components of a gene editing system, an expression vector that includes a coding sequence, and additional reagents and instructions that promote integration of the coding sequence into a tumor genome. The additional reagents may include one or more of a polymerase, a ligase, dNTPs, a co-factor, and a topoisomerase. The kit may include one or more tools for delivering the expression cassette and the gene editing system into a subject. For example, the kit may include a syringe or other surgical tool for delivering the composition to the subject. Optionally, the expression cassette may include a promoter or a transcription factor binding site to increase transcription of the antigen. The kit or the composition may be used in a method of inducing expression of a gRNA in a tumor cell of a subject. The kit or compositions may be used in a method of treating a cancer cell. The kit or compositions may be used in a method treating cancer in a subject. Alternatively, the kit or the composition may be used in conjunction with other compositions to treat cancer in the subject.

According to some aspects, this disclosure provides a method of inducing tumor cell death. The method comprising identifying one or more fusions in tumor DNA obtained from a subject; delivering to said subject a gene editing system, a first vector comprising DNA encoding a guide RNA (gRNA) capable of hybridizing with a common region within a repetitive sequence present in the human genome, and a second vector comprising DNA encoding a Cas-related endonuclease; wherein said gene editing system targets one or more of said fusions; and wherein expression of said gRNA and said Cas-related endonuclease result in cleavage of said tumor DNA. Preferably, said expression control sequence comprises a promoter.

The combined recombinant DNA molecules may be packaged in a vector. The vector may include a gene editing system that integrate the nucleic acid encoding said gRNA and said Cas-related endonuclease, into a genome of a cell. The gene editing system may include a targeting sequence that binds specifically to a target in the genome of said cell. In some embodiments, the targeting sequence may not be found in matched normal sequences from healthy, non-tumor cells of a subject. Healthy cells may be identified as cell not comprising tumor. According to some other embodiments, the targeting sequence may be one or more fusions identified by analyzing tumor DNA obtained from a tumor cell of said subject to identify a sequence of said tumor DNA that is not found in matched normal sequences from healthy, non-tumor cells of said subject. The tumor cell may be identified on account of being taken from tumor tissue of a subject. The tumor cell may be identified by staining the tumor cell with a dye for known surface markers associated with tumorigenesis. In some embodiments, the gene editing system may include a gRNA complimentary to the targeting sequence. The vector may be delivered to said subject. For example, the vector may be delivered via injection. The vector may be carried through a host using a de-activated virus. Upon delivery, the tumor cell expresses said gRNA and further expresses Cas. The gRNA hybridizes to said repetitive element and said Cas-related endonuclease cleaves at least a portion of a sequence of said repetitive element, thereby destroying said tumor cell in said subject.

Certain aspects of the invention rely on targeting fusions. Fusions may be contiguous sequences of DNA formed by the “fusion” of two previously separate contiguous sequences of DNA. In particular, one of the salient abnormalities in the cancer genome is chromosomal rearrangements, which may often result in the joining of two unrelated contiguous sequences of DNA, generally genes, in the chromosome to produce a fusions or fusion genes. Fusions predominately occur in cancer cells. For purposes of the invention, the sequence of the fusion is immaterial once the fusion sequence is identified (e.g., by comparison of cancer genome material with somatic sequence).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of inducing tumor cell death, the method comprising:
identifying one or more fusions in tumor DNA obtained from a subject;
delivering to said subject a gene editing system, a first vector comprising DNA encoding a guide RNA (gRNA) capable of hybridizing with a common region within a repetitive sequence present in the human genome, and a second vector comprising DNA encoding a Cas-related endonuclease; wherein said gene editing system targets one or more of said fusions; and wherein expression of said gRNA and said Cas-related endonuclease result in cleavage of said tumor DNA.

2. The method of claim 1, wherein the gene editing system integrated the first and second vectors into the tumor DNA of a tumor cell in said subject, thereby causing the tumor cell to express the coding sequence of the gRNA and the Cas-related endonuclease.

3. The method of claim 2, wherein said first and second vectors are lentiviral or adeno-associated virus (AAV) vectors.

4. The method of claim 1, wherein the gene editing system includes a targeting sequence that binds specifically to one or more of said fusions in said tumor cell, wherein the target is not found in matched normal sequences from healthy, non-tumor cells of the subject.

5. The method of claim 4, wherein said gene editing system comprises a Cas-related endonuclease and a gRNA, wherein the gRNA includes the targeting sequence.

6. The method of claim 1, wherein said one or more fusions are identified by analyzing said tumor DNA to identify a sequence of said tumor DNA that is not found in matched normal sequences from healthy, non-tumor cells of said subject.

7. The method of claim 5, wherein said analyzing step includes sequencing said tumor DNA to obtain tumor sequences.

8. The method of claim 6, further comprising:
sequencing matched, normal DNA from the healthy, non-tumor cells of said subject to thereby obtain matched normal sequences;
aligning said tumor sequences to said matched normal sequences; and
identifying a fusion as a section of said tumor sequence that does not have an exact match in said matched normal sequences.

9. The method of claim 1, wherein said repetitive sequence is a plurality of repetitive sequences located throughout the human genome and said expressed Cas-related endonuclease and said expressed gRNA cleave each of the plurality of repetitive sequences within said tumor DNA thereby inducing death of said tumor cell.

10. The method of claim 9, wherein one or more of the plurality of repetitive sequences is adjacent a protospacer adjacent motif in said tumor DNA and said expressed gRNA is capable of targeting a portion thereof.

11. The method of claim 9, wherein repetitive sequences is an interspersed retrotransposon sequence.

12. The method of claim 11, wherein said interspersed retrotransposon sequence is a short interspersed nuclear element (SINE) or a long interspersed nuclear element (LINE).

13. The method of claim 11, wherein said SINE is an Alu sequence.

14. The method of claim 11, wherein said LINE is an L1 sequence.

15. The method of claim 1, wherein said Cas-related endonuclease is a Cas9 endonuclease.

16. The method of claim 1, wherein said first and second vectors are lipid nanoparticles.

17. The method of claim 1, wherein said first and second vector each further comprise an expression control sequence operably linked to said encoding DNA.

18. The method of claim 17, wherein said expression control sequence comprises a promoter.

19-35. (canceled)
